SEQUENCE LISTING (7 sequences)

SEQ ID NO: 1 - protein, 14 aa, Lawsonia intracellularis
IDFKAKGVWDFNFE

SEQ ID NO: 2 - protein, 9 aa, Lawsonia intracellularis
FKAKGVWDF

SEQ ID NO: 3 - protein, 23 aa, Lawsonia intracellularis
IDFKAKGVWDFNFEWQQSSFMKG

SEQ ID NO: 4 - protein, 9 aa, Lawsonia intracellularis
GVWDFNFEW

SEQ ID NO: 5 - protein, 9 aa, Lawsonia intracellularis
NFEWQQSSF

SEQ ID NO: 6 - protein, 9 aa, Lawsonia intracellularis
FEWQQSSFM

SEQ ID NO: 7 - protein, 21 aa, Lawsonia intracellularis
MKLGYKISAGFAIGMIMVVLM

(19) United States
(12) Patent Application Publication
(10) Pub. No.: US 2009/0215698 A1
(43) Pub. Date: Aug. 27, 2009

(54) LAWSONIA PROTEIN USEFUL AS A COMPONENT IN SUBUNIT VACCINE AND METHODS OF MAKING AND USING THEREOF

(75) Inventors: Merrill Schaeffer, St. Joseph, MO (US); Jeremy Kroll, Urbandale, IA (US); Michael Roof, Ames, IA (US)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: Boehringer Ingelheim Vetmedica, Inc., Saint Joseph, MO (US)

(21) Appl. No.: 11/911,942

(22) PCT Filed: Apr. 18, 2006
(86) PCT No.: PCT/US06/14705

§ 371 Date: Sep. 16, 2008

Related U.S. Application Data
(60) Provisional application No. 60/672,455, filed on Apr. 18, 2005.

(51) Int. Cl. A61K 38/10; C07K 14/00; C12N 15/11; A61K 31/7088; A61K 38/08

(52) U.S. Cl. 514/13; 530/350; 530/324; 530/328; 530/327; 530/326; 530/325; 514/14; 536/23.1; 514/44 R; 514/16

(57) ABSTRACT

The present invention provides nucleic acid and amino acid sequences useful as the immunogenic portion of vaccines or immunogenic compositions effective for lessening the severity of the clinical symptoms associated with Lawsonia intracellularis infection or conferring protective immunity to an animal susceptible to such infection. Preferred amino acid sequences include at least 9 contiguous amino acids from SEQ ID NOS 1 (IDFKAKGVWDFNNFE), 3 (IDFKAKGVWDFNNFEWQQSSFMKG), or 7 (MKLGYKISAGFAIGMIMVVLM). Thus, the nucleic acid sequences encoding such proteins, or the proteins themselves are included in vaccine compositions, together with veterinary-acceptable carrier and administered to an animal in need thereof.

### RELATED APPLICATIONS

This application is filed under 35 U.S.C. §371, claiming the benefit of international application number PCT/US2006/014705, filed on Apr. 18, 2006, currently pending, which claims the benefit of provisional application Ser. No. 60/672,455, filed on Apr. 18, 2005, now expired, and the teachings and contents of both applications are hereby incorporated by reference.

### SEQUENCE LISTING

This application contains a sequence listing in paper formal and in computer readable format, the teachings and content of which are hereby incorporated by reference.

### BACKGROUND OF THE INVENTION

1. Field of the Invention

The present application is concerned with Lawsonia intracellularis. More particularly, the present application is concerned with immunologically relevant proteins and the nucleic acid sequences encoding those proteins that are capable of invoking an immune response in a host animal. Still more particularly, the present application is concerned with such proteins and their incorporation into an immunogenic composition and its subsequent administration to a host animal. The proteins can be used as a component in a vaccine and the vaccine used to provide a degree of protective immunity against and/or a lessening of the clinical symptoms associated with infection by Lawsonia intracellularis. The present application is also concerned with methods of producing and administering vaccines comprising such nucleic acid sequences or the proteins encoded thereby. Finally, the present application is concerned with diagnostic tests for the detection of Lawsonia intracellularis as well as methods of producing and administering vaccine incorporating

2. Description of the Prior Art

Lawsonia Intracellularis is the causative agent of porcine proliferative interopathy (“PPE”), and it effects virtually all animals, including humans, rabbits, ferrets, hamsters, fox, horses, and other animals as diverse as ostriches and emus. PPE is a group of chronic and acute conditions of widely differing clinical signs, which include death, pale and anemic animals, watery, dark or bright red diarrhea, depression, reduced appetite and reluctance to move, retarded growth, and increased FCR. The bacteria itself is an obligate, intracellular bacterium.

The bacteria associated with PPE have been referred to as “Campylobacter-like organisms.” S. McOrist et al., Vet. Pathol., Vol. 26, 260-264 (1989). Subsequently, the causative bacteria have been identified as a novel taxonomic genus and species, vernacularly referred to as Ileal symbiont (IS) intracellularis. C. Gebhart et al., Int'l. J. of Systemic Bacteriology, Vol. 43, No. 3, 533-538 (1993). More recently, these novel bacteria have been given the taxonomic name Lawsonia (L.) intracellularis. S. McOrist et al., Int'l. J. of Systemic Bacteriology, Vol. 45, No. 4, 820-825 (1995). These three names have been used interchangeably to refer to the same organism as further identified and described herein. Koch's postulates have been fulfilled by inoculation of pure cultures of L. intracellularis into conventionally reared pigs; typical lesions of the disease were produced, and L. intracellularis was reisolated from the lesions. The more common, nonhemorrhagic form of the disease often affects 18- to 36-kg pigs and is characterized by sudden onset of diarrhea. The feces are watery to pasty, brownish, or faintly blood stained. After ˜2 days, pigs may pass yellow fibrinonecrotic casts that have formed in the ileum. Most affected pigs recover spontaneously, but a significant number develop chronic necrotic enteritis with progressive emaciation. The hemorrhagic form is characterized by cutaneous pallor, weakness, and passage of hemorrhagic or black, tarry feces. Pregnant gilts may abort. Lesions may occur anywhere in the lower half of the small intestine, cecum, or colon but are most Frequent and obvious in the ileum. The wall of the intestine is thickened, and the mesentery may be edematous. The mesenteric lymph nodes are enlarged. The intestinal mucosa appears thickened and rugose, may be covered with a brownish or yellow fibrinonecrotic membrane, and sometimes has petechial hemorrhages. Yellow necrotic casts may be found in the ileum or passing through the colon. Diffuse, complete mucosal necrosis in chronic cases causes the intestine to be rigid, resembling a garden hose. Proliferative mucosal lesions often are in the colon but are detected only by careful inspection at necropsy. In the profusely hemorrhagic form, there are red or black, tarry feces in the colon and clotted blood in the ileum. Altogether, L. intracellularis is a particularly great cause of losses in swine herds in Europe as well as in the United States.

L. intracellularis is an obligate, intracellular bacterium which cannot be cultured by normal bacteriological methods on conventional cell-free media and has been thought to require cells for growth. S. McOrist et al., Infection and Immunity, Vol. 61, No. 19, 4286-4292 (1993) and G. Lawson et al., J. of Clinical Microbiology, Vol. 31, No. 5, 1136-1142 (1993) discuss cultivation of L. intracellularis using IEC-18 rat intestinal epithelial cell monolayers in conventional tissue culture flasks. In U.S. Pat. Nos. 5,714,375 and 5,885,823, both of which patents are herein incorporated by reference in their entireties, cultivation of L. intracellularis in suspended host cells was described.

Pathogenic and non-pathogenic attenuated bacteria strains of L. intracellularis are well known in state of the art. For example, WO 96/39629 and WO 05/011731 describe non-pathogenic attenuated strains of L. intracellularis. Further attenuated bacteria strains of L. intracellularis are known from WO 02/26250 and WO 03/00665.

What is needed in the art is a vaccine effective against Lawsonia Intracellularis infection, which provides or confers protective immunity to an animal and/or reduces the severity of clinical symptoms associated with Lawsonia Intracellularis infection. What is further needed are methods of making and administering such vaccines.

### SUMMARY OF THE INVENTION

The present invention overcomes the problems inherent in the prior art and provides a distinct advance in the state of the art. Generally, the present invention describes the identification of proteins or amino acid sequences from Lawsonia intracellularis (hereafter, Lawsonia), which elicit a humoral immune response during the normal course of infection in swine. These proteins, both individually and in combination, will be useful as a component in a protein subunit vaccine that invokes an immune response and provides protective immunity against or a lessening of the clinical symptoms associated with Lawsonia intracellularis infection. The proteins were identified by conventional means of anion exchange separation followed by Western blot using convalescent pig serum. Three proteins were identified and their N-termini were sequenced. These results were then compared with known sequences using BLAST analysis. Of course, these same proteins could be identified by other means by those of skill in the art, including database searching for putative membrane proteins, chromatographic separation of proteins, and other anion exchange methods using gradient conditions that are determined by those of skill in the art. The identified proteins can then be generated by any conventional means and used in a vaccine.

As used herein, the term “L. intracellularis” means the intracellular, curved gram-negative bacteria described in detail by C. Gebhart et al., Int'l. J. of Systemic Bacteriology, Vol. 43, No. 3, 533-538 (1993) and S. McOrist et al., Int'l. J. of Systemic Bacteriology, Vol. 45, No. 4, 820-825 (1995), each of which is incorporated herein by reference in their entireties, and includes but is not limited to the isolates described in WO 96/39629 and WO 05/011731. In particular, the term “L. intracellularis” also means, but is not limited to the isolates deposited under the Budapest Treaty with the American Type Culture Collection, 10801 University Boulevard, Manassas, Va. 20110-2209 and assigned ATCC accession number PTA 4926 or ATCC accession number 55783. Both isolates are described in WO 96/39629 and WO 05/011731, respectively. The term “L. intracellularis” also means, but is not limited to any other L. intracellularis bacteria strain or isolate preferably having the immunogenic properties of at least one of the L. intracellularis strains described in WO 96/39629 and WO 05/011731, in particular having the immunogenic properties of at least one of the isolates deposited under the Budapest Treaty with the American Type Culture Collection, 10801 University Boulevard, Manassas, Va. 20110-2209 and assigned ATCC accession numbers PTA 4926 or ATCC accession number 55783.

A strain or isolate has the “immunogenic properties” of at least one of the L. intracellularis strains described in WO 96/39629 and WO 05/011731, in particular, of the isolates deposited as ATCC accession number PTA 4926 or ATCC accession number 55783, when it is detectable at least with one of the anti-L. intracellularis specific antibodies, described in WO06/01294, in a detection assay that is also described in WO06/01294. Preferably those antibodies are selected from the antibodies having the reference numbers 301:39, 287:6, 268:29, 110:9, 113:2 and 268:18. Preferably, the detection assay is a sandwich ELISA as described in Examples 2 and 3 of WO06/12949, whereas antibody 110:9 is used as an capture antibody and antibody 268:29 is used as conjugated antibody. All antibodies disclosed in WO06/12949 are produced by hybridoma cells, which are deposited at the Centre for Applied Microbiology and Research (CAMR) and European Collection of Cell Cultures (ECACC)”, Salisbury, Wiltshire SP4 0JG, UK, as a patent deposit according to the Budapest Treaty. The date of deposit was May 11, 2004. HYBRIDOMA CELL LINE 110:9 is successfully deposited under ECACC Acc. No. 04092204. HYBRIDOMA CELL LINE 113:2 is successfully deposited under ECACC Acc. No. 04092201. HYBRIDOMA CELL LINE 268:18 is successfully deposited under ECACC Acc. No. 04092202. HYBRIDOMA CELL LINE 268:29 is successfully deposited under ECACC Acc. No. 04092206. HYBRIDOMA CELL LINE 287:6 is successfully deposited under ECACC Acc. No. 04092203. HYBRIDOMA CELL LINE 301:39 is successfully deposited under ECACC Acc. No. 04092205.

Moreover, the term “L. intracellularis” also means any L. intracellularis antigen. The term “L. intracellularis antigen” as used herein means, but is not limited to any composition of matter, that comprises at least one antigen that can induce, stimulate or enhance the immune response against a L. intracellularis-caused infection, when administered to a pig. Preferably, said L. intracellularis antigen is a complete L. intracellularis bacterium, in particular in an inactivated form (a so called killed bacterium), a modified live or attenuated L. intracellularis bacterium (a so called MLB), a chimeric vector that comprises at least an immunogenic amino acid sequence of L. intracellularis, or any other polypeptide or component, that comprises at least an immunogenic amino acid sequence of L. intracellularis. The terms “immunogenic protein”, “immunogenic polypeptide” or “immunogenic amino acid sequence” as used herein, refer to any amino acid sequence which elicits an immune response in a host against a pathogen comprising said immunogenic protein, immunogenic polypeptide or immunogenic amino acid sequence. In particular, an “immunogenic protein”, “immunogenic polypeptide” or “immunogenic amino acid sequence” of L. intracellularis means any amino acid sequence that codes for an antigen which elicits an immunological response against L. intracellularis in a host to which said “immunogenic protein”, “immunogenic polypeptide” or “immunogenic amino acid sequence” is administered.

An “immunogenic protein”, “immunogenic polypeptide” or “immunogenic amino acid sequence” as used herein, includes but is not limited to the full-length sequence of any proteins, analogs thereof, or immunogenic fragments thereof. The term “immunogenic fragment” means a fragment of a protein which includes one or more epitopes and thus elicits the immunological response against the relevant pathogen. Such fragments can be identified using any number of epitope mapping techniques that are well known in the art. See, e.g., Epitope Mapping Protocols in Methods in Molecular Biology, Vol. 66 (Glenn E. Morris, Ed., 1996) Humana Press, Totowa, N.J. (The teachings and content of which are incorporated by reference herein.) For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Iumnunol. 23:709-715. (The teachings and content of which are incorporated by reference herein.) Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; Gardner et al., (1998) 12th World AIDS Conference, Geneva, Switzerland, June 28-Jul. 3, 1998. (The teachings and content of which are incorporated by reference herein.)

An “immunological or immune response” to a composition or vaccine is the development in the host of a cellular and/or antibody-mediated immune response to the composition or vaccine of interest. Usually, an “immune response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of the symptoms associated with host infections as described above.

In addition, the immunogenic and vaccine compositions of the present invention can include one or more veterinary-acceptable carriers. As used herein, “a veterinary-acceptable carrier” includes any and all solvents, dispersion media, coatings, adjuvants, stabilizing agents, diluents, preservatives, antibacterial and antifungal agents, isotonic agents, adsorption delaying agents, and the like.

“Diluents” can include water, saline, dextrose, ethanol, glycerol, and the like. Isotonic agents can include sodium chloride, dextrose, mannitol, sorbitol, and lactose, among others. Stabilizers include albumin and alkalisalts of ethylendiamintetracetic acid, among others.

Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene is glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). John Wiley and Sons, NY, pp 51-94 (1995) and Todd et al., Vaccine 15:564-570 (1997). For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P, 934P and 971P. Most preferred is the use of Cabopol 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others.

Preferably, the adjuvant is added in an amount of about 100 μg to about 10 mg per dose. Even more preferred the adjuvant is added in an amount of about 100 μg to about 10 mg per dose. Even more preferred the adjuvant is added in an amount of about 500 μg to about 5 mg per dose. Even more preferred the adjuvant is added in an amount of about 750 μg to about 2.5 mg per dose. Most preferably, the adjuvant is added in an amount of about 1 mg per dose.

The vaccine composition can further include one or more other immunomodulatory agents such as, e.g., interleukins, interferons, or other cytokines. The vaccine compositions can also include Gentamicin and Merthiolate. While the amounts and concentrations of adjuvants and additives useful in the context of the present invention can readily be determined by the skilled artisan, the present invention contemplates compositions comprising from about 50 ug to about 2000 ug of adjuvant and preferably about 250 ug/ml dose of the vaccine composition. In another preferred embodiment, the present invention contemplates vaccine compositions comprising from about 1 ug/ml to about 60 ug/ml of antibiotics and/or immunomodulatory agents, and more preferably less than about 30 ug/ml of antibiotics and/or immunomodulatory agents.

According to a further embodiment the vaccine is first dehydrated. If the composition is first lyophilized or dehydrated by other methods, then, prior to vaccination, said composition is rehydrated in aqueous (e.g. saline, PBS (phosphate buffered saline)) or non-aqueous solutions (e.g. oil emulsion (mineral oil, or vegetable/metabolizable oil based/single or double emulsion based), aluminum-based, carbomer based adjuvant).

In more detail, one aspect of the present invention provides an immunogenic or vaccine composition comprising an amino acid sequence having at least 9 contiguous amino acids from either of SEQ ID NOS. 1, 3, or 7. Preferably, the sequence having at least 9 contiguous amino acids will be selected from the group consisting of SEQ ID NOS 2, 4, 5, 6, and combinations thereof. Still more preferably, the immunogenic or vaccine composition will comprise SEQ ID NOS 1, 3, or 7. Even more preferably, the antigenic component of the immunogenic or vaccine composition will consist essentially of any one of SEQ ID NOS. 1-7, and combinations thereof. Still more preferably, the amino acid sequence which includes the required contiguous amino acids will be up to 9 amino acids in length, more preferably, up to 14 amino acids in length, still more preferably up to 23 amino acids in length, even more preferably, up to 40 amino acids in length, still more preferably, at least up to 70 amino acids in length, and still more preferably, up to 100 amino acids in length, and still more preferably up to 200 amino acids in length. In preferred forms, the immunogenic or vaccine composition of the present invention will further comprise veterinary-acceptable carriers, as set forth above.

Another aspect of the present invention provides an immunogenic or vaccine composition comprising nucleic acid sequences encoding at least 9 contiguous amino acids from SEQ ID NOS. 1, 3, or 7. Preferably, the sequence having at least 9 contiguous amino acids will be selected from the group consisting of SEQ ID NOS 2, 4, 5, 6, and combinations thereof. Still more preferably, the immunogenic or vaccine composition will comprise the nucleic acid sequences encoding SEQ ID NOS 1, 3, or 7. Even more preferably, the antigenic component of the immunogenic or vaccine composition will consist essentially of the nucleic acid sequences encoding any one of SEQ ID NOS. 1-7, and combinations thereof. In preferred forms, the immunogenic or vaccine composition of the present invention will further comprise veterinary-acceptable carriers, as set forth above. Owing to the degeneracy of the genetic code, it is known that several variations of nucleic acids may encode the same protein. As the encoding of amino acids and the genetic code are both well known in the art, all such variations in nucleic acid sequences that result in the same amino acid are covered by the present invention.

An other aspect of the present invention provides a diagnostic assay utilizing proteins in accordance with the invention. Preferably, the protein is selected from the group consisting of SEQ ID NOS. 1-7, and combinations thereof. Such proteins could be used in an ELISA-based test, Such a protein could also be injected into an animal (e.g. a rabbit) to create an antiserum useful for detecting antibody or antigen. Such assays would be useful in confirming or ruling out Lawsonia infection.

Another aspect of the present invention provides an expression system for expressing proteins useful for purposes of the present invention. Those of skill in the art are familiar with such expression systems. A preferred expression system in this regard will utilize E. coli to express or generate recombinant proteins. Preferably, the E. coli will have nucleic acid sequences inserted therein which encode for proteins, as described above.

In another aspect of the present invention, fusion proteins and chimeras are provided. Preferably, the proteins present or expressed will comprise any one of SEQ ID NOS. 1-7.

Vaccine or immunogenic compositions according to the invention may be administered intramuscularly, intranasally, orally, intradermally, intratracheally, or intravaginally. Preferably, the composition is administered intramuscularly, orally, or intranasally. In an animal body, it can prove advantageous to apply the compositions as described above via an intravenous injection or by direct injection into target tissues. For systemic application, the intravenous, intravascular, intramuscular, intranasal, intraarterial, intraperitoneal, oral, or intrathecal routes are preferred. A more local application can be effected subcutaneously, intradermally, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, the compositions according to the invention may be administered once or several times, also intermittently, for instance on a daily basis for several days, weeks or months and in different dosages.

### BRIEF DESCRIPTION OF THE DRAWINGS

FIG. 1 is a photograph of a Coomassie blue stained gel of AIEX fractions;

FIG. 2 is a photograph of a Western Blot of AIEX fractions wherein anti-LI serum is followed by the conjugate;

FIG. 3 is a Western Blot using the VPM53 MAb;

FIG. 4 is a flow chart illustrating the fractionation of lawsonia proteins;

FIG. 5 is a photograph of a Coomassie blue stained gel of the fractionated proteins from FIG. 4;

FIG. 6 is a photograph of a Western Blot of the respective Lawsonia protein fractions;

FIG. 7 is a Western Blot of two selected Lawsonia proteins on NuPAGE gels; and

FIG. 8 is a photograph of Coomassic stained protein fractions from FIG. 4 on NuPAGE gels.

### DETAILED DESCRIPTION OF THE PREFERRED EMBODIMENTS

The following examples set forth preferred materials and procedures in accordance with the present invention. It is to be understood, however, that these examples are provided by way of illustration only, and nothing therein should be deemed a limitation upon the overall scope of the invention.

### Example 1

This example describes the isolation and sequencing of the protein of the present invention.

### Anionic Exchange Separation

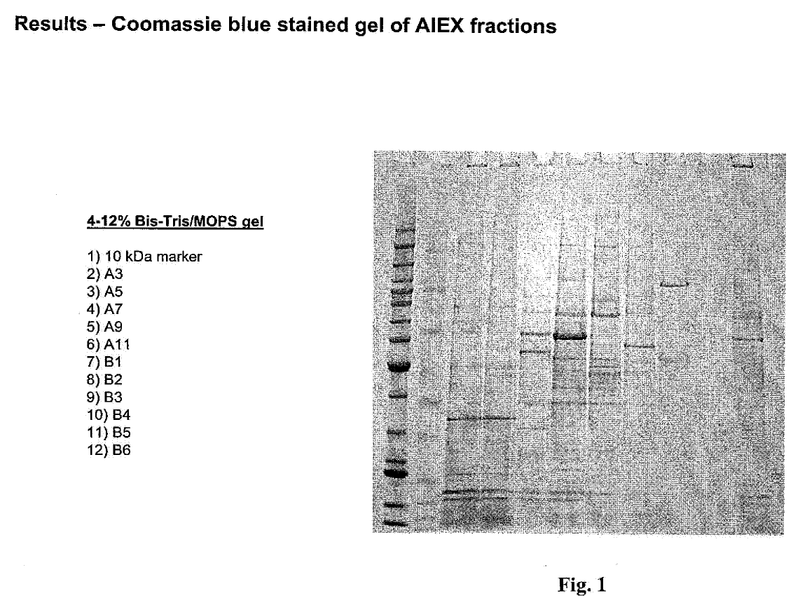

In order to separate proteins from Lawsonia intracellularis (“Lawsonia”), Lawsonia was first grown under standard conditions in a container having 1 L volume using McCoy cells. The extracellular Lawsonia cells were harvested by first filtering the culture through a 5 micron filter in order to remove the McCoy cells and other cell debris. This was then followed by centrifugation sufficient to pellet the bacteria. The supernatant was discarded and the pellet was then washed with in PBS to remove residual media components. After washing, the pellet primarily contained Lawsonia cells. This final preparation of cells was then dissolved in 2 mL solution of 50 mM Tris buffer (pH 8.0), 5 mM 2-mercaptoethanol (“2-ME”), and 8M urea buffer. After extraction for approximately 30 minutes, the mixture was centrifuged for 10 minutes at 20,000×g in order to remove urea-insoluble material. The resulting urea-soluble material was then loaded onto a 1 mL Q Sepharose anion exchange column, where the proteins were separated over a gradient of 0-0.6 M NaCl over 20 column volumes. One milliliter fractions were then collected, and peak fractions were separated in a second dimension following standard SDS-PAGE procedure (4-12% Bis/Tris in MOPS buffer). The resulting gel may be viewed as FIG. 1.

### Western Blot of Fractions Using Convalescent Pig Serum

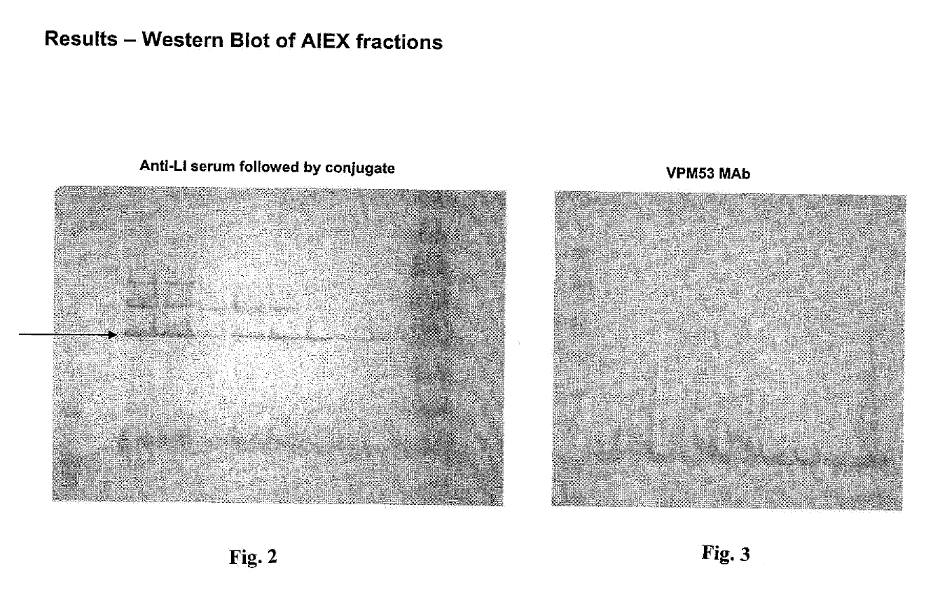

Following the SDS-PAGE, the proteins were then transferred to a PVDF membrane and blotted using swine anti-Lawsonia convalescent serum. The serum was diluted to 1:100 in a TTBS buffer containing a 2% blocking reagent (dry milk). The membrane was maintained at a constant 30V for over an hour using a Novex blot module (Invitrogen, Carlsbad, Calif.). Next, a second blot was done with VPM53 Mab, which was diluted to 1:50. Next, the membrane was washed three times with TTBS. Each wash lasted two minutes. The membrane was then incubated for at least one hour with a secondary antibody. This secondary antibody was goat anti-swine-HRP (KPL, Gaithersburg, Md.), which was diluted to 1:1000 in TTBS+2% dry milk. The membrane was then washed twice for two minutes with TTBS, then washed once for two minutes with PBS. Detection of the protein was accomplished with a OPTI-4CN substrate (Bio-Rad, Hercules, Calif.), which was developed for about 30 minutes, then rinsed with water to stop. The results of the blots may be seen in FIG. 2 and FIG. 3. The resulting protein shown is a ˜52 kDa protein that was detected by the convalescent serum.

### Isolation of Protein for N-Terminal Sequencing

The fractions containing the above-mentioned protein were then concentrated by TCA/acetone precipitation and then suspended in a 1×SDS-PAGE buffer containing 10 mM 2-ME. The proteins were then separated using standard SDS-PAGE procedure (4-12% Bis/Tris in MOPS buffer). The proteins were then transferred from the gel to a PVDF membrane. The membrane was maintained at a constant 30 V for at least one hour using the Novex blot module before being dried completely and stained with an aqueous Coomassie blue stain (Invitrogen, Carlsbad, Calif.). The approximately 52 kDa protein corresponding to that which was detected by Western blot was then excised from the blot using a sterile razor blade. The excised protein was then sent to the Protein Facility at Iowa State University for N-terminal sequencing. The resulting sequence, IDFKAKGVWDFNFE, is designated SEQ ID No. 1.

### Discussion

The N-terminal sequence was utilized to search various databases for homologous sequences. The top hit protein was from Desulfovibrio spp., a closely related organism to Lawsonia. It is likely that this protein has a signal sequence and characteristics of an outer membrane protein, thereby rendering this protein an excellent candidate for incorporation into an immunogenic composition or vaccine operable for eliciting an immune response in swine. Such an immune response will provide a degree of protective immunity against Lawsonia infection.

### Example 2

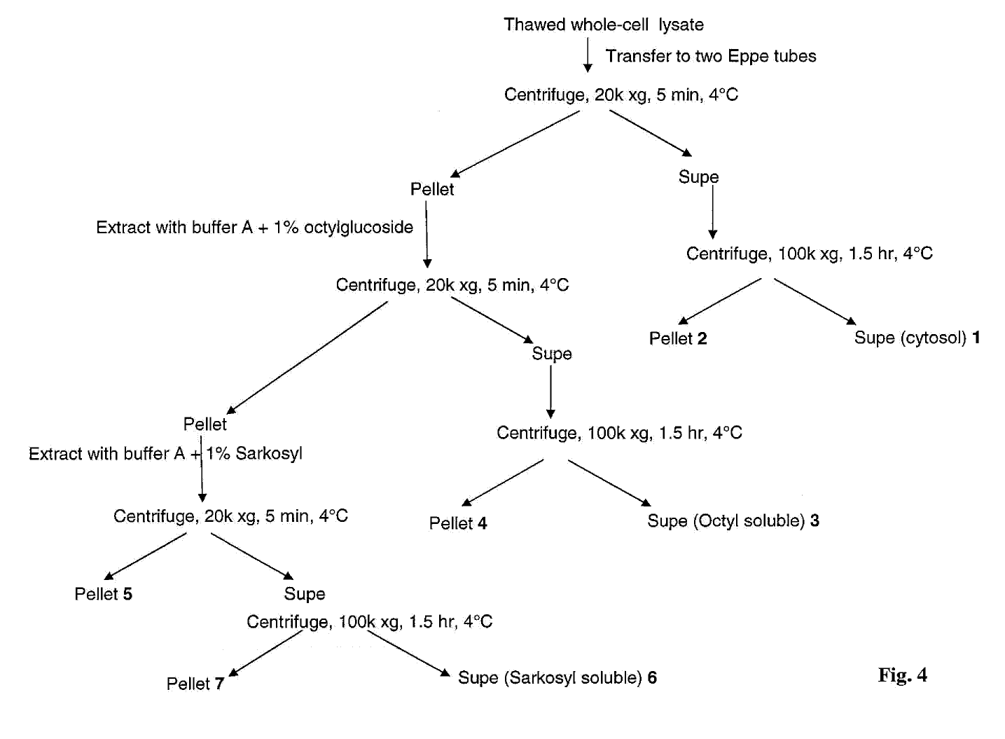

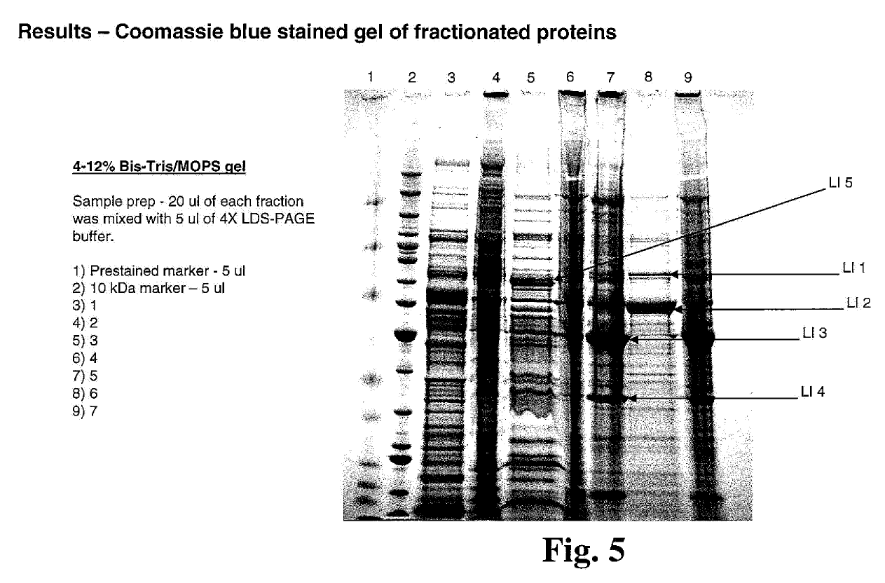

This example describes the isolation and sequencing of a three other proteins of the present invention. Extracellular Lawsonia cells were prepared by filtering the culture through a five μm filter and centrifuging under conditions sufficient to pellet the bacteria. The resulting pellet was suspended in buffer A, which comprised 2.5 ml of 50 mM sodium phosphate, 0.5 M NaCl, and 5 mM 2-ME, at a ph of 7.4). The cells were disrupted through sonication before being subjected to three freeze/thaw three cycles, each comprising one minute pulses with 0.5 second duty cycles for a total often minutes. The sonication step was repeated once more for about five minutes and the resultant mixture (the whole cell lysate) was frozen and stored at −85° C. until it was removed for use. To fractionate the proteins from the whole cell lysate, the lysate was thawed and then transferred to two eppe tubes that were centrifuged for five minutes at 20,000×g at 4° C. this produced a first supernate and a first pellet. The first supernate was centrifuged at 100,000×g at 4° C. for 1.5 hours to produce a second supernate and a second pellet. This second supernate is labeled as Supe (cytosol) 1 in FIG. 4 and the pellet is labeled as Pellet 2 in FIG. 4. The first pellet from the initial centrifugation of the thawed whole cell lysate was extracted with buffer A plus 1% octylglucoside. This was centrifuged for five minutes at 20,000×g at 4° C. to produce a third pellet and supernate. The third supernate was then centrifuged the same as the first supernate in order to produce a fourth supernate product, which is labeled as Supe (Octyl soluble) 3 and a fourth pellet, labeled Pellet 4 in FIG. 4. The third pellet was again extracted with butter A, this time with 1% Sarkosyl before centrifuging at 20,000×g for five minutes at 4° C. This produced a fifth pellet and fifth supernate. The fifth pellet is labeled as Pellet 5 in FIG. 4. The filth supernate was centrifuged in the same manner as the previous supernates in order to produce a sixth supernate, which is labeled in FIG. 4 as Supe (Sarkosyl soluble) 6, and a sixth pellet, which is labeled in FIG. 4 as Pellet 7. Each of the samples obtained in this example were then subjected to Coomassie blue staining, the results of which are shown in FIG. 5. In that figure, lanes 3-9 correspond to fractionated proteins 1-7, as shown in FIG. 4. Fractionated proteins 3, 5, and 6 (Supe 3, Pellet 5, and Supe 6) were then subjected to Western Blot Analysis using convalescent pig serum. The proteins labeled 3, 5, and 6 were transferred from gel to PVDF membrane, which was then subjected to a constant 30 V for at least one hour using a Novex blot module. This was blocked for at least one hour in about 50 ml TTBS plus 2% dry milk (w/v). The TTBS is made by adding 0.05% of freshly prepared Tween 20 to one liter of a 10×TBS solution comprising a filter sterilized mixture of 200 ml of 1 M Tris at a ph of 8, and 292.2 grams NaCl, that has been pH adjusted to 7.4 with HCl and qs to one liter. The membrane was then incubated with a primary antibody (swine anti-Lawsonia intracellularis) 1:100 in TTBS plus 2% dry milk for at least one hour. This was then washed three times for two minutes each time with TTBS. The membrane was then incubated with a secondary antibody (goat anti-swine-HRP, KPL, lot # XD047) 1:1000 in TTBS plus 2% dry milk for at least one hour. This was then washed twice for two minutes each time with TTBS before washing one time for two minutes with 10×PBS. One liter of the 10×PBS solution was made by adding 0.96 grams NaH₂ PO₄ (monobasic) anhydrous, 13.1 grams Na₂HPO₄ (dibasic) anhydrous 87.7 grams NaCl, all of which are dissolved in water and adjusted to a ph of 7.4 and qs to one liter before filter sterilizing. Finally, ten ml of Opti-4 CN lot #99051 was added as the substrate and developed for up to 30 minutes before rinsing with water to stop.

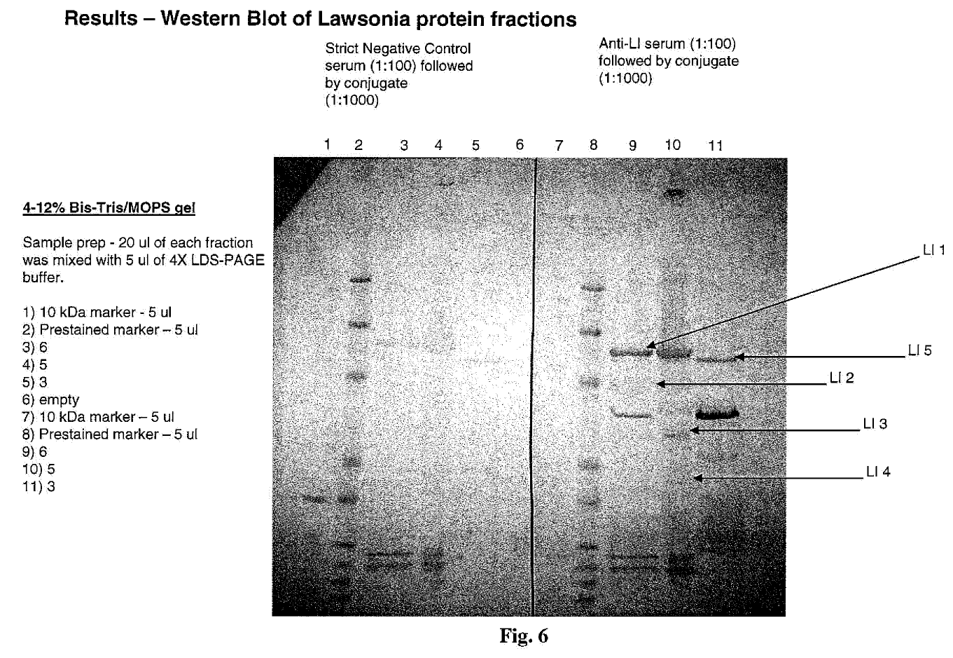

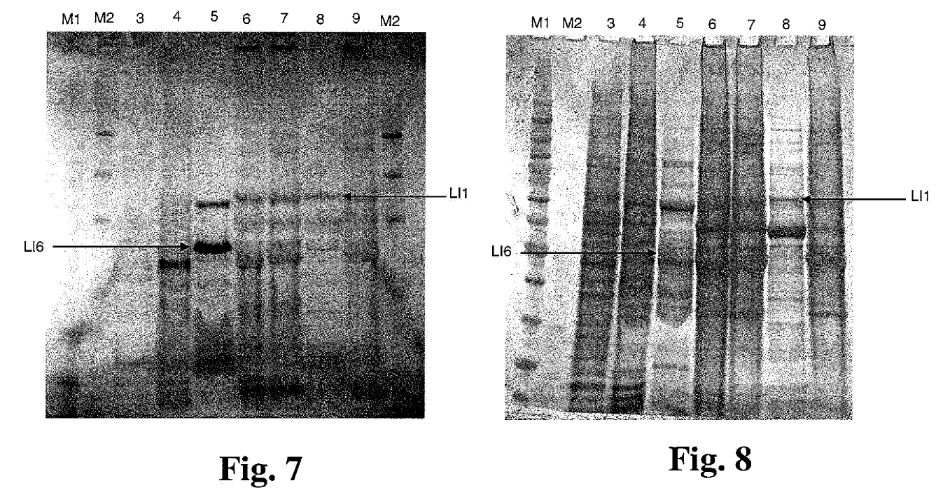

FIG. 6 presents the results of the Western Blot of the respective Lawsonia protein fractions, 3, 5, and 6. Each Western Blot is in 4-12% Bis-Tris/MOPS gel. For the sample prep, 20 microliters of each fraction was mixed with five microliters of 4×LDS-PAGE buffer. Lanes 1-6 contained the strict negative control serum (1:100) followed by the conjugate (1:1000). Lanes 7-11 contained the anti-Lawsonia intracellularis serum (1:100) followed by the conjugate (1:1000). Lane 1 contained the 10 kDa marker (5 microliters), lane 2 contained the prestained marker (5 microliters), lane 3 contained protein fraction 6 (Supe 6), lane 4 contained protein fraction 5 (Pellet 5), lane 5 contained protein fraction 3 (Supe 3), lane 6 was empty, lane 7 contained the 10 kDa marker (5 microliters), lane 8 contained the prestained marker (5 microliters), lane 9 contained protein fraction 6 (Supe 6), lane 10 contained protein fraction 5 (Pellet 5), and lane 11 contained protein fraction 3 (Supe 3). Replicates of fractions 3 and 6 (20 μl each) were run 10 times on 4-12% NuPAGE gels with MOPS buffer for transfer to PVDF membranes. These results are given in FIGS. 7 and 8. In FIG. 7, anti-Lawsonia intracellularis serum (1:50) was followed by the conjugate (1:1000) and lanes 3-9 correspond to fractions 1-7 of FIG. 4. Coomassic stained protein fractions are provided in FIG. 8 where lanes 3-9 correspond to fractions 1-7 from FIG. 4.

The fractionation procedure resulted in fairly distinctive profiles for each protein fraction. In FIG. 6, LI 1 and LI 2 were from Supe 6. These protein fractions are octylglucoside insoluble and sarkosyl soluble and are likely from the cell wall fraction. LI 3 and LI 4 were from Pellet 5. These protein fractions are octyl and sarkosyl insoluble and appear to be membrane proteins. L15 was from Supe 3 and is octyl soluble. This protein fraction is likely from the cell wall.

Of the fractionated proteins, LI 1 and LI 6 were excised from the membrane of FIGS. 6 and 7, and their N-terminals were sequenced. The N-terminal sequence from LI 6, from Supe 3, is designated as SEQ ID NO. 3 and the N-terminal sequence from LI, from Supe 6, is designated as SEQ ID NO. 7.

### Example 3

This example provides sub-sequences or SEQ ID Nos. 1 and 3 that are immunologically relevant and can be used to illicit an immune response against Lawsonia Intracellularis, thereby providing an animal susceptible to Lawsonia Intracellularis infection protective immunity, as well as a lessening of the clinical symptoms associated with infection from Lawsonia Intracellularis.

SEQ ID Nos. 1 and 3 were analyzed for potential epitopes using a SVM and ANN-based CTL epitope prediction tool, as described in Vaccine, 2004 Aug. 13; 22 (23-24): 3195-204, Prediction of CTL Epitopes using QM, SVM, and ANN Techniques, Bhasin M, and Raghava G P, Institute of Microbial Technology, Sector 39A, Chandigarh, India, the teachings and contents of which are incorporated by reference. SEQ ID No. 1 contained 1 epitope, which had a score (ANN/SVM) of 0.82/−0.063950275. This sequence is provided herein as SEQ ID No. 2. SEQ ID No. 3 contained four epitopes, SEQ ID No. 4, which had a score of 0.91/0.68874217, SEQ ID No. 5, which had a score or 0.73/0.55686949, SEQ ID No. 6, which had a score of 0.83/0.17021055, and SEQ ID No. 2.

### Example 4

This example describes the formation of a vaccine. Generally, any one of or a combination of SEQ ID Nos. 1-7 are provided for use as the antigenic portion of a vaccine. Veterinary-acceptable carriers, such as adjuvants, dilulents, and the like will be added to the vaccine and the vaccine will be administered in any conventional manner.

We claim:

1. A composition comprising:
an amino acid sequence having less than 200 amino acids and having therein at least 9 contiguous amino acids from an amino acid sequence selected from the group consisting of SEQ ID NOS. 1, 3, and 7.

2. The composition of claim 1, said amino acid sequence having less than 70 amino acids.

3. The composition of claim 1, said at least nine contiguous amino acids being selected from the group consisting of SEQ ID NOS. 2, 4, 5, 6, and combinations thereof.

4. The composition of claim 1, further comprising a veterinary acceptable carrier.

5. The composition of claim 1, said composition being in a formulation acceptable for intramuscular, oral, or nasal administration.

6. The composition of claim 1, said composition being effective for conferring protective immunity against Lawsonia intracellularis infection or for lessening the severity of clinical symptoms associated with Lawsonia intracellularis infection.

7. A nucleic acid sequence encoding an amino acid sequence having less than 200 amino acids and having therein at least 9 contiguous amino acids from an amino acid sequence selected from the group consisting of SEQ ID NOS. 1, 3, and 7.

8. The nucleic acid sequence of claim 7, said nucleic acid sequence encoding an amino acid sequence having less than 70 amino acids.

9. The nucleic acid sequence of claim 7, said at least nine contiguous amino acids being selected from the group consisting of SEQ ID NOS. 2, 4, 5, 6, and combinations thereof.

10. The nucleic acid sequence of claim 7, further comprising a veterinary acceptable carrier.

11. The nucleic acid sequence of claim 7, said nucleic acid sequence being in a formulation acceptable for intramuscular, oral, or nasal administration.

12. The nucleic acid sequence of claim 7, said sequence being effective for conferring protective immunity against Lawsonia intracellularis infection or for lessening the severity of clinical symptoms associated with Lawsonia intracellularis infection, when administered to an animal susceptible to Lawsonia intracellularis infection.

13. A fusion protein having therein at least 9 contiguous amino acids from an amino acid sequence selected from the group consisting of SEQ ID NOS. 1, 3, and 7.

14. The fusion protein of claim 13, said amino acid sequence having less than 70 amino acids.

15. The fusion protein of claim 13, said at least nine contiguous amino acids being selected from the group consisting of SEQ ID NOS. 2, 4, 5, 6, and combinations thereof.

16. The fusion protein of claim 13, further comprising a veterinary acceptable carrier.

17. The fusion protein of claim 13, said fusion protein being in a formulation acceptable for intramuscular, oral, or nasal administration.

18. The fusion protein of claim 13, said fusion protein being effective for conferring protective immunity against Lawsonia intracellularis infection or for lessening the severity of clinical symptoms associated with Lawsonia intracellularis infection, when administered to an animal susceptible to such infection.
